(19) United States
(12) Patent Application Publication
(10) Pub. No.: US 2009/0191140 A1
(43) Pub. Date: Jul. 30, 2009

(54) MAKE-UP POWDER, AND PROCESS FOR ITS PREPARATION

(75) Inventor: Piero ORIGO, Mozzanica (IT)

Correspondence Address: YOUNG & THOMPSON, 209 Madison Street, Suite 500, ALEXANDRIA, VA, 22314, US

(73) Assignee: ART COSMETICS S.R.L., Mozzanica (IT)

(21) Appl. No.: 12/358,562

(22) Filed: Jan. 23, 2009

(30) Foreign Application Priority Data
Jan. 25, 2008 (IT) MI2008A112

(51) Int. Cl. A61K 8/26

(52) U.S. Cl. 424/63

(57) ABSTRACT

Disclosed are cosmetic powders comprising:
a) a first phase comprising mixed silicates of aluminium and alkaline metals (mica) and polysaccharides;
b) a second phase comprising a mixture of natural polysaccharide gums.

This invention relates to a cosmetic composition in powder form, characterised by the absence of a binder phase consisting of fatty and/or oily and/or waxy and/or hydrocarbon and/or silicone and/or fluorinated substances.

### PRIOR ART

Cosmetic compositions in powder form, and in particular cosmetic compositions formulated for the colouring of the face, body and hair, commonly known as “make-up”, are made in different cosmetic forms such as free powder, compact powder, baked powder, cast powder and extruded powder. In order to optimise the characteristics of the formulation, it is common practice to use in the formulation of these powders a set of raw materials technically described as the “binder phase”, consisting of substances and/or mixtures of fatty, oily, waxy, hydrocarbon, silicone or fluorinated substances of animal, plant, extracted or synthetic origin, as more particularly described below and as disclosed, for example, in EP 890356 and in EP 1053741.

Said binder phase is essential to ensure the cohesion of the powders so that they can be compacted or extruded and in any event to guarantee a suitable cosmetic form for their transport, sale and/or use.

The binder phase is also essential to cause the cosmetic to adhere to the skin or hair and thus guarantee the long duration of the product after application and a pleasant feel and pay off intensity during its application.

According to the state of the art, a binder phase is technically essential to make a cosmetic in powder form; however, its use also presents contraindications.

Not all the powders and powder mixtures used possess the same ability to absorb the binder phase; similarly, the binder phase is diffused differently through the powders that make up the formulation. This considerably limits the possibility of making a homogenous formulation.

In many cases the binder phase presents incompatibilities with the skin, and in particular has an occlusive action, with adverse effects on the health of the skin.

### DESCRIPTION OF THE INVENTION

Surprisingly, it has been found that if a particular type of ingredients in powder form is used, mixed together in appropriate ratios using a suitable manufacturing technique, cosmetic powders can be obtained which, compared with known formulations, present exceptional, advantageous properties such as:
resistance to compacting, baking and extrusion;
a pleasant feel;
pay off on skin and hair;
persistence after application;
total absence of occlusive power towards the skin;
a homogenous structure.

The powdered products according to the invention may be in free, compact, cast, baked or extruded form, and can be used on the body and/or face and/or hair.

The powders according to the invention do not contain fatty, oily, waxy, hydrocarbon, silicone or fluorinated substances or mixtures of substances of animal, plant, extractive or synthetic origin.

The powders according to the invention comprise:
a) a first phase comprising mixed silicates of aluminium and alkaline metals (mica) and polysaccharides;
b) a second phase comprising a mixture of natural polysaccharide gums.

The powders according to the invention will also contain dyes, preservatives, fillers, pearls and other conventional components required to achieve the desired cosmetic effect.

The polysaccharides in phase a) are preferably selected from starches, and more preferably corn starch. The ratio between mica and starch can vary within a wide range, and will be between 1:99 and 99:1, and preferably between 40:60 and 60:40.

The percentage of phase a) contained in the final formulation ranges between 0.5% and 99%, and preferably between 25 and 35%.

The natural polysaccharide gums are preferably selected from xanthan gum mixed with polyalcohols having a number of carbon atoms ranging between 2 and 6 and a number of hydroxy groups ranging between 2 and 6, preferably glycerol, in a variable ratio ranging between 1:99 and 99:1, and preferably between 15:85 and 45:55.

The percentage of phase a) contained in the final formulation ranges between 0.5% and 99%, but preferably between 4 and 15%.

The powders according to the invention are prepared by a process which involves:

1) mixing said first phase with the other powdered components contained in the formulation such as dyes, pearls, preservatives and fillers;

2) dispersing said second phase in water at a concentration ranging between 0.5% and 99%, and preferably between 40% and 60%;

3) adding the dispersion obtained in 2) to the mixture obtained in 1); 4) drying in the oven at a temperature ranging between 0° C. and 150° C., preferably 60° C., and at a pressure ranging between 10 and 1520 mmHg, and preferably 760 mmHg.

Phase 1) can be mixed using fan grinders, hammer grinders, step grinders, ball grinders or micronisers, for example.

The invention is described in greater detail in the following Example.

### EXAMPLE

First phase

| | |
|---|---|
| Mica | 12.00% |
| Corn starch | 18.00% |
| Nylon 12 | 6.70% |
| Preservatives | 0.30% |
| Red iron oxide | 12.00% |
| Yellow iron oxide | 10.00% |
| Black iron oxide | 10.00% |
| Pearl | 10.00% |

Second phase

| | |
|---|---|
| Water | 13.00% |
| Xanthan gum | 2.00% |
| Glycerol | 6.00% |

1. Cosmetic powders comprising:
a) a first phase comprising mixed silicates of aluminium and alkaline metals (mica) and polysaccharides;
b) a second phase comprising a mixture of natural polysaccharide gums.

2. Powders as claimed in claim 1, wherein
the polysaccharides in phase a) are selected from starches, preferably corn starch.

3. Powders as claimed in claim 1, wherein the natural polysaccharide gums are selected from xanthan gum mixed with polyalcohols having a number of carbon atoms ranging between 2 and 6 and a number of hydroxy groups ranging between 2 and 6, preferably glycerol.

4. Process for preparation of the powders as claimed in claim 1 which comprises:
1) mixing said first phase, comprising mixed silicates of aluminium and alkaline metals (mica) and polysaccharides, with other powdered components contained in the formulation;
2) dispersing said second phase, comprising a mixture of natural polysaccharide gums, in water at a concentration ranging between 0.5% and 99%, and preferably between 40% and 60%;
3) adding the dispersion obtained in 2) to the mixture obtained in 1); 4) drying in the oven at a temperature ranging between 0° C. and 150° C., preferably 60° C., and at a pressure ranging between 10 and 1520 mmHg, and preferably 760 mmHg.

5. Process for preparation of the powders as claimed in claim 2 which comprises:
1) mixing said first phase, comprising mixed silicates of aluminium and alkaline metals (mica) and polysaccharides, with other powdered components contained in the formulation;
2) dispersing said second phase, comprising a mixture of natural polysaccharide gums, in water at a concentration ranging between 0.5% and 99%, and preferably between 40% and 60%;
3) adding the dispersion obtained in 2) to the mixture obtained in 1);
4) drying in the oven at a temperature ranging between 0° C. and 150° C., preferably 60° C., and at a pressure ranging between 10 and 1520 mmHg, and preferably 760 mmHg.
